(19) United States
(12) Patent Application Publication
(10) Pub. No.: US 2005/0222093 A1
(43) Pub. Date: Oct. 6, 2005

(54) Methods for restoring cognitive function following systemic stress

(76) Inventors: Rodney Pearlman, El Granada, CA (US); Ken Tempero, Wayzata, MN (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 11/136,272

(22) Filed: May 23, 2005

Related U.S. Application Data
(63) Continuation of application No. 10/099,537, filed on Mar. 15, 2002.
(60) Provisional application No. 60/275,937, filed on Mar. 15, 2001.
(60) Provisional application No. 60/293,375, filed on May 24, 2001.

(51) Int. Cl. A61K 31/66

(52) U.S. Cl. 514/114

(57) ABSTRACT

The invention provides methods for treating cognitive decline that is associated with systemic stress.

### CROSS-REFERENCE TO RELATED APPLICATIONS AND CLAIM OF PRIORITY

This application claims priority under 35 U.S.C. 119(e) from U.S. Provisional Application No. 60/275,937 filed Mar. 15, 2001, and from U.S. Provisional Application No. 60/293,375 filed May 24, 2001, which applications are incorporated herein by reference.

### BACKGROUND OF THE INVENTION

Cognitive decline in an individual, i.e., a decrease in the mental processes related to, e.g., thinking, reasoning, learning, memory, or judgment, has been associated with the occurrence of a traumatic event such as cardiac surgery. Cognitive dysfunction is also recognized as a response associated with use of benzodiazepine, and sudden, unexpected violent trauma, e.g., military combat, natural disasters, or serious accidents.

Advances in techniques for anesthesia, surgery, and the protection of organs have resulted in substantial reductions in mortality associated with cardiac surgery. However, the incidence of cognitive decline has changed little over the past 15 years (Newman et al., New England Journal of Medicine, 344, 396 (2001)). Newman et al. report the relatively high prevalence and persistence of cognitive decline following CABG. The authors suggest that cognitive decline after CABG is present in as many as three quarters of patients at the time of discharge from the hospital and a third of patients after six months. The clinical and financial implications of these problems can be profound, since prolonged hospitalization and an increased use of resources are associated with major and even minor neurobehavioral declines, not to mention the effects that cognitive decline has on the patient's quality of life.

Benzodiazepines, one of the most commonly prescribed classes of drugs, are primarily used to treat anxiety and insomnia. As tranquilizers, they are often used as sedatives before some surgical and medical procedures. In fact, patients who have not been given a benzodiazepine preoperatively have complained of remembering surgery. Thus, benzodiazepines are often specifically administered to patients prior to surgery because they cause anterograde amnesia. However, one of the problems associated with acute as well as long term use of these drugs, particularly in the elderly, is memory loss. Since aging is inherently associated with some degree of increasing memory impairment and the elderly population is prone to insomnia, prescribing benzodiazepine is problematic.

Problems of memory, cognition, and concentration have also been reported in patients suffering from post-traumatic stress disorder (P.T.S.D.) (see generally “What is Post-Traumatic Stress Disorder” Fact Sheet, National Center for P.T.S.D., http://www.ncptds.org/facts/general Jun. 12, 2000). It is noteworthy that P.T.S.D., at times, may not manifest itself clinically for twenty or more years following the traumatic event.

Therefore, there is currently a need for methods for preventing and/or treating the short and/or long term cognitive decline associated with systemic stress.

### SUMMARY OF INVENTION

Systemic stress is associated with events such as an environmental event, e.g., relocation of residence or hospitalization (short or long term), especially in the elderly, or exposure to environmental toxin; a health problem, e.g., an illness such as hypertension; a medical treatment, e.g., surgery or drug treatment such as anesthesia; and a sudden, unexpected violent trauma; e.g., military combat, a natural disaster, or a serious accident. The present invention provides a method of treating cognitive decline associated with systemic stress comprising administering to a mammal in need of such therapy, an effective amount of a cognitive enhancing agent.

The invention provides the use of cognitive enhancing agent for the manufacture of a medicament useful for the treatment of cognitive decline associated with systemic stress.

### BRIEF DESCRIPTION OF THE DRAWINGS

FIG. 1 depicts a cognitive enhancing agent (compound 1) which is particularly useful in the methods of the invention.

FIG. 2 depicts a cognitive enhancing agent (compound 2) which is particularly useful in the methods of the invention.

FIG. 3 depicts a cognitive enhancing agent (compound 3) which is particularly useful in the methods of the invention.

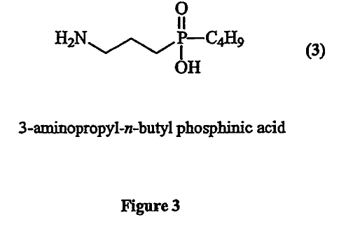

### DETAILED DESCRIPTION OF THE INVENTION

I. Definitions

“Systemic stress” is meant to refer to sub-acute or chronic stress, as well as the acute stress associated with a traumatic event or events (anticipated or unanticipated). Stress is considered as any adverse condition or influence which tends to disrupt the normal, steady functioning of the body and its parts, and generally is adverse to its well-being. By “sub-acute stress” or “chronic stress” is meant continued or prolonged exposure to a source of stress, i.e., stressor, which can lead to an elevated blood level of cortisol, a major stress hormone. Stressors are varied and include positive as well as negative events, and they may be external or internal. Examples of stressors include, but are not limited to, environmental stressors; health-related stressors, e.g., health problems, medical treatment; major trauma (e.g. cerebrovascular and traumatic brain injury); and sudden, unexpected violent trauma.

“Systemic stress due to an environmental event” refers to the stress associated with an environmental stressor, e.g., an exposure to a toxic substance such as carbon monoxide or a radioactive substance, as well as to the stress caused by the disruption and/or removal from a normal setting and/or routine. Examples of the latter include the relocation of a residence, and both short- and long-term hospitalization, all of which are especially problematic in the elderly.

By “systemic stress due to a health problem” is meant the systemic stress caused by an illness or condition, such as hypertension, high blood pressure, stroke, cardiac disease, cardiac failure, pulmonary insufficiency, atrial dysfunction, panic attacks/anxiety, insomnia, depression, chronic fatigue, myalgic encephalitis, an allergy, asthma, ulcerative colitis, Crohn's disease, irritable bowel syndrome, an ulcer, inadequate diet, a vitamin deficiency, a metabolic disturbance, thyroid disease, anoxia, depression, diabetes, hypoglycemia, hyperglycemia, hyperinsulinemia, vasculopathy, encephalitis, insomnia, including a sleep deficit and abnormal sleep patterns, brain tumor, epilepsy, subdermal hematoma, and an infection such as a brain or meningeal infection, syphilis, and Lyme Disease.

By “systemic stress due to a medical treatment” refers to the systemic stress due to an event such as, but not limited to, surgery and/or the awareness of need for or awareness of an impending surgery, e.g., cardiac surgery and neurosurgery, as well as a treatment such as electroconvulsive therapy. As used herein, the term “cardiac surgery” includes coronary artery bypass grafting, as well as any other procedure that requires the use of extracorporeal circulation (i.e. a diversion of blood flow through a circuit located outside the body). In addition, the term “medical treatment” includes the administration of a drug, e.g., anesthesia or drug therapy administered prior to surgical treatment, e.g., pretreatment with a benzodiazapine.

“Stress due to sudden, unexpected violent trauma” includes the stress associated with post traumatic stress disorder (P.T.S.D.) or post traumatic stress syndrome (P.T.S.S.), as well as a stress associated with any sudden, unexpected violent trauma. “Sudden, unexpected violent trauma” includes, but is not limited to, witnessing or experiencing a traumatic or life-threatening event, e.g., military combat, a natural disaster, a terrorist incident, a serious accident, a riot, or a violent personal assault, e.g., being mugged, a shooting, e.g., a school, playground, or gang-related shooting.

“Major trauma” refers to abuse, e.g., physical and/or emotional abuse, e.g., childhood abuse, a dysfunctional family, an addiction, co-dependence, substance abuse, including alcohol abuse, and drug abuse, e.g., an addiction to an antidepressant, an analgesic, a sedative, as well as the occurrence of a stroke or seizure, e.g., an epileptic seizure.

As used herein, the term “cognitive decline associated with systemic stress” includes any diminution (whether in time or scope) in the cognitive ability or functionality of a mammal, e.g., a human, that begins or occurs within a short period of time following or results from an incident of stress on the mammal. Cognitive ability or functionality includes both conscious and unconscious mental activities and/or processes.

“Cognitive decline” is meant to include “memory loss”, i.e., any disruption related to learning and memory. A “disruption relating to learning and memory” refers to any impairment associated with memory formation and/or memory recall.

“Memory” can be, for example, short-term memory, long-term memory, explicit memory, i.e., memory for a conscious fact, e.g., the memory of a specific event, or implicit or procedural memory, i.e., memory relating to an “unconsciously” performed task, e.g., riding a bicycle.

As used herein, the term “cognitive enhancing agent” includes any agent or agents, used alone or in combination, useful for increasing the cognitive ability of a mammal. The term includes hormones, such as estrogen; herbal treatments, including Ginko, Ginko biloba, Huperizia serrata, Panex ginseng, Kava-kava, Kavain, and Gota Kola; amino acids, such as L-Glutamine, L-Taurine, L-Tyrosine and N-Acetyl Carnitine (NAC); co-enzymes, including co-enzyme Q; DHEA; nitric oxide; acetylcholinesterase inhibitors, such as heptylphysostigmine and tetrahydroacridine (THA; tacrine); muscarinic agonists, including oxotremorine; inhibitors of angiotensin-converting enzyme, such as octylramipril, captopril, ceranapril, enalapril, lisinopril, fosinopril and zofenopril; centrally-acting calcium channel blockers; nimodipine; nootropic agents, including piracetam; and compounds which bind to the GABA_{β}-receptor and are found to act as antagonists on the GABA_{β}-receptor, including those compounds disclosed in U.S. Pat. No. 5,190,933.

The term cognitive enhancing agent also includes agents disclosed in U.S. Pat. Nos. 6,169,108; 6,156,787; 6,156,761; 6,153,606; 6,143,773; 6,143,722; 5,985,616; 5,840,729; 5,763,458; 5,723,464; 5,723,103; 5,693,642; 5,670,477; 5,610,154; 5,604,198; 5,585,374; 5,530,013; 5,449,682; 5,444,073; 5,430,033; 5,401,744; 5,399,566; 5,385,894; 5,348,955; 5,324,729; 5,312,820; 5,308,846; 5,292,741; 5,292,726; 5,281,614; 5,278,068; 5,726,054; 5,264,439; 5,260,285; 5,256,667; 5,252,574; 5,250,521; 5,246,944; 5,240,938; 5,238,942; 5,233,501; 5,212,195; 5,208,240; 5,202,328; 5,202,322; 5,198,438; 5,187,179; 5,187,159; 5,183,810; 5,177,095; 5,177,074; 5,175,164; 5,166,206; 5,162,340; 5,162,325; 5,157,040; 5,128,327; 5,124,335; 4,904,658; 4,859,666; 4,826,843; 4,668,687; 6,169,112; 6,100,255; 6,066,636; 6,051,340; 6,043,255; 6,037,352; 5,990,132; 5,977,138; 5,962,494; 5,958,967; 5,955,470; 5,952,349; 5,935,958; 5,910,501; 5,889,006; 5,885,608; 5,883,096; 5,866,585; 5,843,469; 5,827,847; 5,804,580; 5,786,508; 5,731,348; 5,721,223; 5,712,302; 5,708,030; 5,695,774; 5,691,365; 5,643,923; 5,639,775; 5,629,333; 5,629,312; 5,594,001; 5,574,055; 5,543,426; 5,541,208, 5,532,242; 5,519,055; 5,508,287; 5,498,623; 5,486,526; 5,470,868; 5,470,856; 5,468,763; 5,464,843; 5,459,161; 5,446,051; 5,434,177; 5,424,301; 5,409,948; 5,403,845; 5,362,860; 5,362,860; 5,326,781; 5,326,770; 5,318,967; 5,318,966; 5,308,851; 5,296,507; 5,278,162; 5,260,324; 5,244,909; 5,299,407; 5,227,394; 5,208,260; 5,204,482; 5,200,414; 5,190,954; 5,175,166, 5,087,633; 5,061,721; 5,015,645; 5,013,737; 5,013,737; 5,011,849; 4,997,832; 4,985,437; 4,906,626; 4,897,399; 5,204,342; 5,135,930; 5,439,930; 5,439,930; 5,190,933; 5,064,819; 5,300,679; 5,013,863; and 4,845,115.

The term cognitive enhancing agent also includes agents disclosed by Richards et al., Mol. Pharm., 58, 577 (2000); Meneses, Neuroscience & Behavioral Reviews, 23, 1111 (1999); Reisner et al., Neuroscience Letters, 274, 187 (1999); Friedman et al., Biological Psychiatry, 46, 1243 (1999); McGaughy et al., Psychopharmacology, 144, 175 (1999); Hilgert et al., Neuroscience Letters, 263, 193 (1999); Lewis et al., Phytotherapy Res., 13, 59 (1999); Yates et al., J. Pharm. & Exp. Therapeutics, 289, 1151 (1999); Levin and Simon, Psychopharmacology, 138; 217 (1998); O'Neill et al., Progress in Neuro-Psychopharmacology & Biological Psychiatry, 22, 665 (1998); Pirotte et al., J. Medicinal Chemistry, 41, 2946 (1998); Paroczai et al., Brain Research Bulletin, 45, 475 (1998); Allain and Bentue-Ferrer, European Neurology, 39, 39 (1998); Schneider et al., Annals of Neurology, 43, 311 (1998); Halbreich, Psychopharmacology Bulletin, 33, 281 (1997); Genkova-Papazova et al., Pharmacology, Biochemistry & Behavior, 56, 583 (1997); Bojanova et al., Methods & Findings in Experimental & Clinical Pharmacology, 19, 93 (1997); Lin et al., J Medicinal Chemistry, 40, 385 (1997); Secades and Frontera, Methods & Findings in Experimental & Clinical Pharmacology, 17, 2 (1995); de Saint Hilaire et al., Pharmacology, Biochemistry & Behavior, 52, 819 (1995); Ono et al., Chemical & Pharmaceutical Bulletin, 43, 1492 (1995); Ono et al., Chemical & Pharmaceutical Bulletin, 43, 1488 (1995); Ono et al., Chemical & Pharmaceutical Bulletin 43, 1483 (1995); Sullivan et al., Proceedings of the Western Pharmacology Society, 38, 127 (1995); Villalobos et al., J of Medicinal Chemistr, 38, 2802 (1995); Scapecchi et al., Bioorganic & Medicinal Chemistry, 2, 1061 (1994); Genkova-Papazova et al., Pharmacology, Biochemistry & Behavior, 49, 849 (1994); Baxter et al., Neurobiology of Aging, 15, 207 (1994); Delumeau et al., J of Neural Transmission, 41, 259 (1994); Domeney, J Psychiatry & Neuroscience 19, 46 (1994); Windsor et al., J of Chromatography, 619, 315 (1993); Wilkerson et al., J. Medicinal Chemistry, 36, 2899 (1993); Doods et al., Life Sciences, 52, 497 (1993); Ceda et al., Hormone & Metabolic Research, 24, 119 (1992); Di Trapani and Fioravanti, Clinica Terapeutica, 137, 403 (1991); Waters, Canadian J. Of Neurological Sciences 15, 249 (1988); Porsolt et al., Psychopharmacology, 95, 291 (1988); Bompani and Scali, Current Medical Research & Opinion, 10, 99 (1986); Shih and Pugsley, Life Sciences, 36, 2145 (1985); and Butler et al., J of Medicinal Chemistry, 24, 346 (1981).

A specific cognitive enhancing agent useful in the methods of the invention is a compound described in U.S. Pat. Nos. 5,190,933; 5,064,819; 5,300,679; 5,051,524; and 5,013,863. For example, a compound of Formula (I): 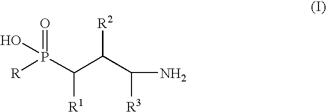
wherein R denotes an aliphatic, cycloaliphatic, cycloaliphatic-aliphatic or araliphatic radical having 2 or more carbon atoms, and wherein one of the groups R¹, R² and R³ represents hydrogen or an aliphatic, cycloaliphatic, araliphatic or aromatic radical, another one of R¹, R² and R³ is hydrogen or, in the case of R¹ and R² is hydroxy, and the remaining one of R¹, R² and R³ is hydrogen, or wherein R denotes methyl, R¹ denotes hydrogen or hydroxy, R² denotes an aromatic radical and R³ represents hydrogen; or a pharmaceutically acceptable salt thereof. Preferably, for a compound of Formula (I), R is different from 1,1-di(C₁-C₄-alkoxy)-C₁-C₅-alkyl, if one of R¹, R² and R³ represents hydrogen, C₁-C₈-alkyl, C₃-C₆-cycloalkyl, phenyl optionally substituted by halogen, C₁-C₄-alkyl, C₁-C₄-alkoxy and/or trifluoromethyl or C₇-C₁₀-phenylalkyl optionally substituted in the phenyl moiety by halogen, C₁-C₄-alkyl, C₁-C₄-alkoxy and/or trifluoromethyl and the other two of R¹, R² and R³ are hydrogen. Preferably, for a compound of Formula (I), R is different from ethyl if R² represents hydroxy and R¹ and R³ are hydrogen. Preferably, for a salt of a compound of Formula (I), when R denotes an unsubstituted aliphatic, cycloaliphatic or araliphatic hydrocarbon radical, R¹ and R³ denote hydrogen and R² is hydrogen or alkyl, the counter ion is not an alkali metal or ammonium.
For a Compound of Formula (I), the Following Definitions and Preferred Values Apply:

Aliphatic radicals R are, for example, alkyl groups that may be interrupted by one or two mutually spaced atoms selected from oxygen and sulfur and/or substituted by halogen or hydroxy, such as alkyl, alkyl mono-, di- or poly-substituted by halogen and/or hydroxy, alkyl being interrupted by one or two mutually spaced atoms selected from oxygen and sulfur or alkyl being interrupted by one or two mutually spaced atoms selected from oxygen and sulfur and substituted by halogen and/or hydroxy, alkenyl groups that may be mono-, di- or poly-substituted by halogen and/or hydroxy, such as lower alkenyl or lower alkenyl substituted by halogen and/or hydroxy, or alkynyl groups, such as lower alkynyl. Aliphatic radicals R¹, R² or R³ are, for example, lower alkyl groups.

Cycloaliphatic radicals R are, for example, cycloalkyl groups that may be interrupted by one or two mutually spaced atoms selected from oxygen and sulfur and/or substituted by hydroxy, such as cycloalkyl, cycloalkyl being interrupted by one or two mutually spaced atoms selected from oxygen and sulfur or cycloalkyl substituted by hydroxy. Cycloaliphatic radicals R¹, R² or R³ are, for example, cycloalkyl groups.

Cycloaliphatic-aliphatic radicals R are, for example, cycloalkyl-lower alkyl groups that may be interrupted by one or two mutually spaced atoms selected from oxygen and sulfur and/or substituted by hydroxy and/or lower alkylthio, such as cycloalkyl-lower alkyl, cycloalkyl-lower alkyl being interrupted by one or two mutually spaced atoms selected from oxygen and sulfur or cycloalkyl-lower alkyl substituted in the cycloalkyl moiety by hydroxy or lower alkylthio and/or in the alkylene moiety by hydroxy.

Araliphatic radicals R and/or R¹, R² or R³ are, for example, phenyl-lower alkyl or naphthyl-lower alkyl radicals that may be substituted in the aryl ring by halogen, lower alkyl, lower alkoxy and/or trifluoromethyl and/or in the lower alkylene moiety by hydroxy, such as phenyl-lower alkyl, phenyl-(1-hydroxy)-lower alkyl, naphthyl-lower alkyl or phenyl-lower alkyl substituted in the phenyl moiety by halogen, lower alkyl, lower alkoxy and/or trifluoromethyl.

Aromatic radicals R¹, R² or R³ are, for example, phenyl, naphthyl or phenyl substituted by halogen, lower alkyl, lower alkoxy and/or trifluoromethyl.

In compounds of Formula (I) the group R is bonded to the P-atom via a carbon atom.

Alkyl, alkenyl and alkynyl R may contain up to and including 14, preferably 12 carbon atoms and are represented by lower alkyl, lower alkenyl and lower alkynyl. Alkyl R may also be a C₈-C₁₄-, e.g. a C₈-C₁₂-alkyl, such as an octyl, nonyl, decyl, undecyl or dodecyl group, e.g. a decyl or dodecyl group.

Alkyl or alkenyl mono-, di- or poly-substituted by halogen and/or hydroxy is represented by mono- or dihydroxy-lower alkyl, hydroxy-lower alkenyl, mono-, di- or polyhalogeno-lower alkyl, mono-, di- or polyhalogeno-lower alkenyl, mono-, di- or polyhalogeno-lower hydroxyalkyl and mono-, di- or polyhalogeno-lower hydroxyalkenyl.

Alkyl being interrupted by one or two atoms selected from oxygen and sulfur is represented by lower alkoxy-lower alkyl, lower alkylthio-lower alkyl, lower alkanesulfinyl-lower alkyl, lower alkanesulfonyl-lower alkyl, lower alkoxy-lower alkoxy-lower alkyl, di-lower alkoxy-lower alkyl, di-lower alkylthio-lower alkyl, and lower alkoxy-lower alkylthio-lower alkyl.

Alkyl being interrupted by one or two atoms selected from oxygen and sulphur and substituted by hydroxy and/or halogen is represented by lower alkoxy-(hydroxy)lower alkyl and lower alkoxy-(halogeno)lower alkyl.

Cycloalkyl is represented by C₃-C₈-cycloalkyl.

Cycloalkyl substituted by hydroxy is represented by 1-hydroxy-C₃-C₈-cycloalkyl.

Cycloalkyl and cycloalkyl in cycloalkyl-lower alkyl, in either case, being interrupted by one or two atoms selected from oxygen and sulfur is represented by oxa-C₃-C₈-cycloalkyl, thia-C₃-C₈-cycloalkyl, dioxa-C₃-C₈-cycloalkyl, dithia-C₃-C₈-cycloalkyl and oxathia-C₃-C₈-cycloalkyl.

Cycloalkyl-lower alkyl substituted in the cycloalkyl moiety by hydroxy and/or lower alkylthio and/or in the alkylene moiety by hydroxy is represented by lower alkylthiocycloalkyl-lower alkyl, cycloalkyl-(hydroxy)lower alkyl and lower alkylthiocycloalkyl-(hydroxy)lower alkyl.

The term “lower” in connection with organic radicals or compounds respectively, if not defined explicitly otherwise, defines such with up to and including 7, preferably up to and including 4, carbon atoms.

Lower alkyl R is represented by C₂-C₇-alkyl, especially by C₃-C₇-alkyl, e.g. propyl, isopropyl, butyl, isobutyl, sec.-butyl, tert.-butyl, (2-methyl)butyl, hexyl or heptyl. Lower alkyl other than R denotes, for example, C₁-C₄-alkyl, e.g. methyl, ethyl, propyl, isopropyl, butyl or tert.-butyl.

Lower alkenyl denotes, for example, C₃-C₇-alkenyl, preferably C₃-C₅-alkenyl, carrying the double bond in a higher than the α, β-position, and is e.g. 2-propenyl(allyl), but-3-en-1-yl, (2-methyl)prop-2-en-1-yl(isobutenyl) or (5-methyl)but-2-en-1-yl, but may also carry the double bond in α, β-position and may be, for example, vinyl, prop-1-enyl or but-1-enyl, or may be a C₆- or C₇-alkenyl, such as a hexenyl or heptenyl, group.

Lower alkynyl denotes, for example, C₃-C₇ alkynyl, preferably C₃-C₅-alkynyl, carrying the triple bond in a higher than the α, β-position and is e.g. 2-propynyl(propargyl), but-3-yn-1-yl, but-2-yn-1-yl or pent-3-yn-1-yl.

C₃-C₈-Cycloalkyl preferably has 3 to 6 ring carbon atoms and thus is C₃-C₆-cycloalkyl, e.g. cyclopropyl, cyclobutyl, cyclopentyl or cyclohexyl.

C₃-C₈-Cycloalkyl-lower alkyl preferably has 3 to 6 ring and 1 to 4 chain carbon atoms and is, for example, C₃-C₆-cycloalkyl-C₁-C₄-alkyl, such as cyclopropylmethyl, cyclobutylmethyl or cyclohexylmethyl.

Mono- or dihydroxy-lower alkyl preferably carries one of the hydroxy groups in α-position and is for example, α-hydroxy-C₂-C₇-alkyl, such as α-hydroxy-C₂-C₄-alkyl, e.g. 1-hydroxyethyl, 2-(2-hydroxy)propyl, 1-hydroxybutyl, 2-(2-hydroxy)butyl or 1-(1-hydroxy-2-methyl)propyl, or α,β-dihydroxy-C₂-C₇-alkyl, such as 1,2-dihydroxy-prop-2-yl, but may also carry a single hydroxy group in a higher than the α-position and denote, for example, α-, γ- or Δ-hydroxy-C₂-C₇-alkyl, e.g. 3-hydroxypropyl or 2-, 3- or 4-hydroxybutyl.

Hydroxy-lower alkenyl preferably carries the hydroxy group in α-position and the double bond in a higher than the α, β-position and is, for example, corresponding α-hydroxy-C₃-C₅-alkenyl, e.g., 1-hydroxybut-2-enyl.

Mono-, di- or polyhalogeno-lower alkyl is for example, mono-, di- or trifluoro-C₂-C₅-alkyl, e.g., 3,3,3-trifluoropropyl, 4,4,4-trifluorobutyl, 1- or 2-fluorobutyl or 1,1-difluorobutyl.

Mono-, di- or polyhalogeno-lower alkenyl is, for example, mono-, di- or trifluoro-C₃-C₅-alkenyl, e.g., 2-fluorobut-2enyl.

Mono-, di- or polyhalogeno-lower hydroxyalkyl and mono-, di- or polyhalogeno-lower hydroxyalkenyl preferably carries the hydroxy group in α-position and the halogen atom(s) in a higher than the α-position and is, for example, corresponding mono-, di- or trifluoro-α-hydroxy-C₂-C₇-alkyl or mono-di- or trifluoro-C₃-C₇-alkenyl, e.g., 2-fluoro-1-hydroxy-butyl, 2-fluoro-1-hydroxy-but-2-en-1-yl or 4,4,4-trifluoro-1-hydroxy-butyl.

Lower alkoxy-lower alkyl preferably has up to 10 carbon atoms and is, for example, C₁-C₄-alkoxy-C₁-C₄-alkyl, such as C₁-C₃-alkoxy-C₁-C₃-alkyl, e.g., methoxymethyl, ethoxymethyl, 2-methoxyethyl, 2-ethoxyethyl, 3-methoxypropyl or 1- or 2-methoxybutyl.

Lower alkoxy is, for example, C₁-C₄-alkoxy, e.g., methoxy, ethoxy, isopropoxy, propoxy, butoxy, sec.-butoxy or tert.-butoxy.

Lower alkoxy-lower alkoxy-lower alkyl is, for example, C₁-C₄-alkoxy-C₂-C₄-alkoxy-C₁-C₄-alkyl, e.g., 2-methoxyethoxymethyl.

Lower alkylthio-lower alkyl preferably has up to 10 carbon atoms and is, for example, C₁-C₄-alkylthio-C₁-C₄-alkyl, such as C₁-C₃-alkylthio-C₁-C₃-alkyl, e.g., methylthiomethyl, ethylthiomethyl, 2-methylthioethyl, 2-ethylthioethyl or 3-methylthiopropyl.

Lower alkanesulfinyl- and lower alkanesulfonyl-lower alkyl preferably has up to 10 carbon atoms and is, for example, C₁-C₄-alkanesulfinyl- or C₁-C₄-alkanesulfonyl-C₁-C₄-alkyl, e.g., ethanesulfinylmethyl or ethanesulfonylmethyl.

Di-lower alkoxy-lower alkyl preferably has up to 15 carbon atoms totally and is, for example, di-C₁-C₄-alkoxy-C₁-C₃-alkyl, such as di-C₁-C₃-alkoxy-C₁-C₃-alkyl, e.g., dimethoxymethyl, diethoxymethyl, dipropyloxymethyl, 1,1- or 2,2-diethoxyethyl, dissopropyloxymethyl, di-n-butoxymethyl or 3,3-dimethoxypropyl.

Di-lower alkylthio-lower alkyl preferably has up to 15 carbon atoms totally and is, for example, di-C₁-C₄-alkylthio-C₁-C₄-alkyl, such as di-C₁-C₃-alkylthio-C₁-C₃-alkyl, e.g., dimethylthiomethyl, diethylthiomethyl or 1,1- or 2,2-dimethylthioethyl.

Lower alkoxy-(hydroxy)lower alkyl is, for example C₁-C₄-alkoxy-C₁-C₇-(hydroxy)alkyl, e.g., 2-(2-hydroxy-3-methoxy)propyl.

Lower alkoxy-(halogeno)lower alkyl is, for example C₁-C₄-alkoxy-C₁-C₇-(halogeno)alkyl, e.g., 1-(2-fluoro-1-methoxy)butyl.

Hydroxy-C₃-C₈-cycloalkyl is, for example, 1-hydroxy-C₃-C₆-cycloalkyl, e.g., 1-hydroxycyclobutyl.

Oxa- or thia-C₃-C₈-cycloalkyl preferably has 2 to 6 ring carbon atoms is, for example, 2-oxacyclopropyl(oxiranyl), 2- or 3-oxacyclobutyl(oxetanyl), 2- or 3-thiacyclobutyl(thietanyl), 2- or 3-oxacylcopentyl(tetrahydrofuranyl), 2- or 3-thiacyclopentyl(thiolanyl) or 2-oxacyclohexyl(tetrahydropyranyl).

Dioxa-C₃-C₈-cycloalkyl preferably has 3 to 5 ring carbon atoms and carries those two oxygen atoms in 1,3-position to each other, and represents e.g., 1,3-dioxolan-2-yl or 1,3-dioxan-2yl.

Dithia-C₃-C₈-cycloalkyl preferably has 3 to 5 ring carbon atoms and carries those two sulfur atoms in 1,3-position to each other and represents, e.g., 1,3-dithiolan-2-yl or 1,3-dithioxan-2-yl. Oxathio-C₃-C₈-cycloalkyl is, for example 1,3-oxathiolan-2-yl or 1,3-oxathioxan-2-yl.

C₃-C₈-Cycloalkyl-(hydroxy)lower alkyl preferably has 3 to 6 ring and 1 to 4 chain carbon atoms and is, for example, cyclo-C₃-C₆-alkyl-C₁-C₄-alkyl, e.g., 1-cyclopropyl-1-hydroxymethyl or 1-hydroxy-1-cyclobutylmethyl. Lower alkylthiocycloalkyl-(hydroxy) lower alkyl is, for example, 1-hydroxy-1-(2-methylthiocyclopropyl).

Halogen, as a substituent of aromatic and/or araliphatic radicals R¹, R² or R³, is preferably chloro, but may also be fluoro, bromo or iodo.

A phenyl or naphthyl group may have one or more than one, preferably one or two of the same or different substituents as defined hereinbefore. Phenyl- or naphthyl-lower alkyl is, e.g., benzyl, naphth-2-ylmethyl, 1- or 2-phenylethyl or 2- or 3-phenylpropyl, each optionally substituted as described hereinbefore.

Salts of a compound of a Formula (I) are particularly pharmaceutically acceptable salts thereof, such as the corresponding addition salts with acids, as well as the salts with bases. Suitable acids for the formation of acid addition salts are, for example, mineral acids, such as hydrochloric, hydrobromic, sulphuric or phosphoric acid, or organic acids, such as organic sulphonic acids, for example, benzenesulphonic, 4-toluenesulphonic or methanesulphonic acid, and organic carboxylic acids, such as acetic, lactic, palmitic, stearic, malic, maleic, fumaric, tartaric, ascorbic or citric acid. Salts with bases are, for example, alkali metal or alkaline earth metal salts, such as sodium, potassium, calcium or magnesium salts, or ammonium salts, such as those with ammonia or suitable organic amines, e.g., diethylamine, di-(2-hydroxyethyl)-amine or tri-(2-hydroxyethyl)-amine. The compounds of the Formula I may also form inner salts.

Depending on the presence of asymmetric carbon atoms, the compounds of Formula (I) may be in the form of mixtures of isomers, particularly racemates, or in the form of pure isomers, particularly optical antipodes.

A preferred compound of Formula (I) useful in the methods of the invention is a compound wherein R has 2 or more carbon atoms and denotes alkyl, alkenyl, alkynyl, alkyl or alkenyl mono-, di- or poly-substituted by halogen and/or hydroxy, alkyl being interrupted by one or two mutually spaced atoms selected from oxygen and sulfur, alkyl being interrupted by one or two mutually spaced atoms selected from oxygen and sulfur and substituted by halogen and/or hydroxy, cycloalkyl, cycloalkyl substituted by hydroxy, cycloalkyl being interrupted by one or two mutually spaced atoms selected from oxygen and sulfur, cycloalkyl-lower alkyl, cycloalkyl-lower alkyl substituted in the cycloalkyl moiety by hydroxy or lower alkylthio and/or in the alkylene moiety by hydroxy, cycloalkyl-lower alkyl being interrupted by one or two mutually spaced atoms selected from oxygen and sulfur in the cycloalkyl moiety, phenyl-lower alkyl, naphthyl-lower alkyl or phenyl- or naphthyl lower alkyl ring substituted by halogen, lower alkyl, lower alkoxy and/or trifluoromethyl or naphthyl-lower alkyl, and/or chain-substituted by hydroxy and wherein one of the groups R¹, R² and R³ represents hydrogen, lower alkyl, cycloalkyl, phenyl or naphthyl, phenyl or naphthyl substituted by halogen, lower alkyl, lower alkoxy and/or trifluoromethyl, phenyl-lower alkyl or phenyl lower alkyl substituted in the phenyl moiety by halogen, lower alkyl, lower alkoxy and/or trifluoromethyl, another one of R¹, R² and R³ is hydrogen or, in the case of R¹ and R², is hydroxy and the remaining one of R¹, R² and R³ is hydrogen; or a pharmaceutically acceptable salt thereof.

Another preferred compound of Formula (I) useful in the methods of the invention is a compound wherein R has 2 or more carbon atoms and is lower alkyl, lower alkenyl, lower alkynyl, alkyl being interrupted by one or two mutually spaced atoms selected from oxygen, sulfur and cycloalkyl, cycloalkyl being interrupted by one or two mutually spaced atoms selected from oxygen and sulfur, cycloalkyl or cycloalkyl-lower alkyl being interrupted by one or two mutually spaced atoms selected from oxygen and sulfur in the cycloalkyl moiety, and wherein one of the groups R¹, R² and R³ represents hydrogen, lower alkyl, cycloalkyl, phenyl, phenyl substituted by halogen, lower alkyl, lower alkoxy and/or trifluoromethyl, phenyl lower alkyl or phenyl lower alkyl substituted in the phenyl moiety by halogen, lower alkyl, lower alkoxy and/or trifluoromethyl, or one of R¹ and R² is hydroxy, and the remaining two of R¹, R² and R³ are hydrogen; or a pharmaceutically acceptable salt thereof.

Another preferred compound of Formula (I) useful in the methods of the invention is a compound wherein R has 2 or more carbon atoms and is lower alkyl, lower alkenyl, lower alkynyl, a cycloalkyl, hydroxycycloalkyl, cycloalkyl-lower alkyl, cycloalkyl-(hydroxy)lower alkyl or lower alkylthiocycloalkyl-(hydroxy)lower alkyl group having 3 to 6 ring carbon atoms, mono- or dihydroxy-lower alkyl, hydroxy-lower alkenyl, mono-, di- or polyhalogeno-lower alkyl, mono-, di- or polyhalogeno-lower alkenyl, mono-, di- or polyhalogeno-(hydroxy)lower alkyl, mono-, di- or polyhalogeno-(hydroxy)lower alkenyl, lower alkoxy-lower alkyl, lower alkylthio-lower alkyl, lower alkanesulfinyl-lower alkyl, lower alkanesulfonyl-lower alkyl, di-lower alkoxy-lower alkyl, di-lower alkylthio-lower alkyl, lower alkoxy-(hydroxy)lower alkyl, lower alkoxy-(halogeno)lower alkyl, phenyl-lower alkyl, phenyl-lower alkyl mono- or disubstituted, in the phenyl moiety, by halogen, lower alkyl, lower alkoxy and/or trifluoromethyl, naphthyl-lower alkyl, oxa- or thiacycloalkyl having 2 to 6 ring carbon atoms, or dioxa-, oxathia- or dithiacycloalkyl having 3 to 5 ring carbon atoms, and wherein one of R¹, R², R³ represents hydrogen, lower alkyl, cycloalkyl having 3 to 6 ring carbon atoms, phenyl, phenyl mono- or disubstituted by halogen, lower alkyl, lower alkoxy and/or trifluoromethyl, phenyl-lower alkyl or phenyl-lower alkyl mono- or disubstituted by halogen, lower alkyl, lower alkoxy and/or trifluoromethyl, another one of R¹, R² and R³ is hydrogen or, in the case of R¹ and R², is hydroxy, and the remaining one of R¹, R² and R³ is hydrogen; or a pharmaceutically acceptable salt thereof.

Another preferred compound of Formula (I) useful in the methods of the invention is a compound wherein R has 2 or more carbon atoms and is lower alkyl, lower alkenyl, lower alkynyl, a cycloalkyl, hydroxycycloalkyl, cycloalkyl-lower alkyl, cycloalkyl-(hydroxy)lower alkyl or lower alkylthiocycloalkyl-(hydroxy)lower alkyl group having 3 to 6 ring carbon atoms, hydroxy-lower alkyl, hydroxy-lower alkenyl, mono-, di- or polyhalogeno-lower alkyl, mono-, di- or polyhalogeno-lower alkenyl, mono-, di- or polyhalogeno-(hydroxy)lower alkyl, mono-, di- or polyhalogeno-(hydroxy)lower alkenyl, phenyl-lower alkyl phenyl-lower alkyl mono- or disubstituted, in the phenyl moiety, by halogen, lower alkyl, lower alkoxy and/or trifluoromethyl or naphthyl-lower alkyl, and wherein one of the groups R¹, R² and R³ represents hydrogen, lower alkyl, cycloalkyl, phenyl, phenyl substituted by halogen, lower alkyl, lower alkoxy and/or trifluoromethyl, phenyl lower alkyl or phenyl lower alkyl substituted in the phenyl moiety by halogen, lower alkyl, lower alkoxy and/or trifluoromethyl, another one of R¹, R² and R³ is hydrogen or, in the case of R¹ and R² is hydroxy, and the remaining one of R¹, R² and R³ is hydrogen; or a pharmaceutically acceptable salt thereof.

Another preferred compound of Formula (I) useful in the methods of the invention is a compound wherein R has 2 or more carbon atoms and is, lower alkenyl or lower alkynyl, and wherein one of the groups R¹, R² and R³ represents hydrogen, lower alkyl, cycloalkyl, phenyl, phenyl substituted by halogen, lower alkyl, lower alkoxy and/or trifluoromethyl, phenyl lower alkyl or phenyl lower alkyl substituted in the phenyl moiety by halogen, lower alkyl, lower alkoxy and/or trifluoromethyl, or one of R¹ and R² is hydroxy; and the remaining two of R¹, R² and R³ are hydrogen; or a pharmaceutically acceptable salt thereof.

Another preferred compound of Formula (I) useful in the methods of the invention is a compound wherein R is represented by lower alkoxy-lower alkyl, lower alkylthio-lower alkyl, lower alkanesulfinyl-lower alkyl, lower alkanesulfonyl-lower alkyl, di-lower alkoxy-lower alkyl, di-lower alkylthio-lower alkyl, lower alkoxy-(hydroxy)lower alkyl, lower alkoxy-(halogeno)lower, oxa- or thiacycloalkyl having 2 to 6 ring carbon atoms, or dioxa- or dithiacycloalkyl having 3 to 5 ring carbon atoms, and wherein one of the groups R¹, R² and R³ represents hydrogen, lower alkyl, cycloalkyl, phenyl, phenyl substituted by halogen, lower alkyl, lower alkoxy and/or trifluoromethyl, phenyl lower alkyl or phenyl lower alkyl substituted in the phenyl moiety by halogen, lower alkyl, lower alkoxy and/or trifluoromethyl, another one of R¹, R² and R³ is hydrogen or, in the case of R¹ and R², is hydroxy, and the remaining one of R¹, R² and R³ is hydrogen, provided that, if one of R¹ and R² is hydrogen, lower alkyl, cycloalkyl, phenyl, phenyl substituted by halogen, lower alkyl, lower alkoxy and/or trifluoromethyl, phenyl-lower alkyl or phenyl-lower alkyl substituted in the phenyl moiety by halogen, lower alkyl, lower alkoxy and/or trifluoromethyl, and the other two of R¹, R² and R³ are hydrogen, R is different from 1,1-diC₁-C₄-alkoxy)-C₁-C₅-alkyl; or a pharmaceutically acceptable salt thereof.

Another preferred compound of Formula (I) useful in the methods of the invention is a compound wherein R is represented by lower alkoxy-lower alkyl, lower alkylthio-lower alkyl, di-lower alkoxy-lower alkyl, di-lower alkylthio lower alkyl, lower alkoxy-lower alkylthio-lower alkyl, oxacycloalkyl, thiacycloalkyl, dioxacycloalkyl and dithiacycloalkyl, and wherein one of the groups R¹, R² and R³ represents hydrogen, lower alkyl, cycloalkyl, phenyl, phenyl substituted by halogen, lower alkyl, lower alkoxy and/or trifluoromethyl, phenyl lower alkyl or phenyl lower alkyl substituted in the phenyl moiety by halogen, lower alkyl, lower alkoxy and/or trifluoromethyl, or one of R¹ and R² is hydroxy; and the remaining two of R¹, R² and R³ are hydrogen; or a pharmaceutically acceptable salt thereof.

Another preferred compound of Formula (I) useful in the methods of the invention is a compound wherein R has any meaning defined hereinbefore, and wherein one of the groups R¹, R² and R³ represents hydrogen, lower alkyl, phenyl or phenyl substituted by halogen or lower alkyl, and the remaining two of R¹, R² and R³ are hydrogen; or a pharmaceutically acceptable salt thereof.

Another preferred compound of Formula (I) useful in the methods of the invention is a compound wherein R is lower alkyl having 2 or more carbon atoms, lower alkenyl or lower alkynyl, R² represents hydrogen, lower alkyl, phenyl or phenyl substituted by halogen or lower alkyl and R¹ and R³ are hydrogen; or a pharmaceutically acceptable salt thereof.

Another preferred compound of Formula (I) useful in the methods of the invention is a compound wherein R is lower alkoxy-lower alkyl or mono- or dihydroxy-lower alkyl, R² represents hydrogen, lower alkyl, phenyl or phenyl substituted by halogen or lower alkyl and R¹ and R³ are hydrogen; or a pharmaceutically acceptable salt thereof.

Another preferred compound of Formula (I) useful in the methods of the invention is a compound wherein R is C₂-C₁₂-alkyl, such as ethyl, butyl, isobutyl, pentyl or isopentyl, C₂-C₇-alkenyl, such as but-3-enyl, C₂-C₇-alkynyl, such as pent-3-ynyl, mono- or dihydroxy-C₂-C₇-alkyl, such as 2-(2-hydroxy)propyl, 2-(1,2-dihydroxy)propyl, 2-(2-hydroxy)butyl or 1-hydroxybutyl, mono-, di- or trihalogeno-α-hydroxy-C₃-C₇-alkyl, such as 1-hydroxy-4,4,4-trifluorobutyl, α-saturated mono-, di- or trihalogeno-α-hydroxy-C₃-C₇-alkenyl, such as 1-hydroxy-2-fluoro-but-2-enyl, C₁-C₄-alkoxy-C₁-C₄-alkyl, such as 2-ethoxyethyl, di-C₁-C₄-alkoxy-C₁-C₄-alkyl, such as diethoxymethyl, α-hydroxy-C₃-C₆-cycloalkyl, such as 1-hydroxycyclobutyl, C₃-C₆-cycloalkyl-C₁-C₄-alkyl, such as cyclopropylmethyl, C₃-C₆-cycloalkyl-αhydroxy-C₁-C₃-alkyl, such as 1-cyclobutyl-1-hydroxymethyl, or 1-C₁-C₄-alkylthiocycloalkyl-α-hydroxy-C₁-C₄-alkyl, such as (1-methylthiocyclopropyl)(1-hydroxy)methyl, R² represents hydrogen, hydroxy, C₁-C₄-alkyl, such as methyl, phenyl or phenyl substituted by halogen, such as chloro, or C₁-C₄-alkyl, such as methyl and R¹ and R³ are hydrogen or one of R¹ and R² denotes hydroxy and the other one as well as R³ represents hydrogen; or a pharmaceutically acceptable salt thereof.

A more preferred compound of Formula (I) useful in the methods of the invention is a compound wherein R either is C₂-C₇-alkyl, C₂-C₇-alkenyl or C₂-C₇-alkynyl or denotes C₁-C₄-alkoxy-C₁-C₄-aklyl or di-C₁-C₄-alkoxy-C₁-C₄-alkyl or denotes α-, β-, γ- or Δ-hydroxy-C₂-C₇-alkyl or α,β-dihydroxy-C₂-C₇-alkyl, R² represents hydrogen, lower alkyl, phenyl or phenyl substituted by halogen or lower alkyl, and R¹ and R³ are hydrogen; or a pharmaceutically acceptable salt thereof.

Another more preferred compound of Formula (I) useful in the methods of the invention is a compound wherein R denotes C₂-C₇-alkyl, such as ethyl, butyl, isobutyl, pentyl or isopentyl, α-saturated C₃-C₇-alkenyl, such as but-3-enyl, α-saturated C₃-C₇-alkynyl, such as pent-3-ynyl, α-, β-, γ-, or Δ-hydroxy-C₂-C₇-alkyl, such as 2-(2-hydroxy)propyl or 1-hydroxybutyl, α,β-dihydroxy-C₂-C₄-alkyl, such as 2-(1,2-dihydroxy)propyl, mono-, di- or trifluoro-α-hydroxy-C₃-C₇-alkyl, such as 1-hydroxy-4,4,4-trifluorobutyl, α-saturated mono-, di- or trihalogen-α-hydroxy-C₃-C₇-alkenyl, such as 1-hydroxy-2-fluorobut-2-enyl, C₁-C₄-alkoxy-C₁-C₄-alkyl, such as 2-ethoxyethyl, di-C₁-C₄-alkoxy-C₁-C₄-alkyl, C₃-C₆-cycloalkyl-C₁-C₄-alkyl, such as cyclopropylmethyl, α-hydroxy-C₃-C₆-cycloalkyl, such as 1-hydroxycylobutyl, or C₃-C₆-cycloalkyl-α-hydroxy-C₁-C₄-alkyl, such as 1-cyclopropyl-1-hydroxymethyl, and R¹, R² and R³ represent hydrogen; or a pharmaceutically acceptable salt thereof.

Another more preferred compound of Formula (I) useful in the methods of the invention is a compound wherein R is C₂-C₇-alkyl, C₂-C₇-alkenyl or C₂-C₇-alkynyl, or C₁-C₄-alkoxy-C₁-C₄-alkyl or di-C₁-C₄-alkoxy-C₁-C₄-alkyl and R¹, R² and R³ are hydrogen; or a pharmaceutically acceptable salt thereof.

Another more preferred compound of Formula (I) useful in the methods of the invention is a compound wherein R is C₃-C₇-alkyl and R¹, R² and R³ are hydrogen; or a pharmaceutically acceptable salt thereof.

A most preferred compound of Formula (I) useful in the methods of the invention is the compound 3-amino-2-(4-chlorophenyl)-propyl(diethoxymethyl)phosphinic acid; 3-amino-2-hydroxy-propyl(diethoxymethyl)phosphinic acid; 3-aminopropyl(n-butyl)phosphinic acid; 3-aminopropyl(diethoxymethyl)phosphinic acid; 3-aminopropyl(t-butyl)phosphinic acid; 3-aminopropyl(n-propyl)phosphinic acid; 3-aminopropyl(ethyl)phosphinic acid; 3-aminopropyl(cyclohexyl)phosphinic acid; 3-aminopropyl(isobutyl)phosphinic acid; 3-aminopropyl(n-hexyl)phosphinic acid; 3-aminopropyl(allyl)phosphinic acid; 3-aminopropyl(n-pentyl)phosphinic acid; 3-aminopropyl(n-heptyl)phosphinic acid; 3-aminopropyl(but-3-enyl)phosphinic acid; 3-aminopropyl(n-decyl)phosphinic acid; 3-aminopropyl(isopentyl)phosphinic acid; 3-aminopropyl(cyclopropylmethyl)phosphinic acid; (1-methyl-3-aminopropyl)(n-butyl)phosphinic acid; 3-aminopropyl(pent-3-ynyl)phosphinic acid; 3-aminopropyl(but-3-ynyl)phosphinic acid; 3-aminopropyl(2-ethoxyethyl)phosphinic acid; 3-aminopropyl(2-methylbutyl)-phosphinic acid; 3-aminopropyl-(3-ethoxypropyl)-phosphinic acid; 3-aminopropyl(3-methoxypropyl)phosphinic acid; 3-aminopropyl(but-2-ynyl)phosphinic acid; 3-aminopropyl[2-(2-ethoxyethoxy)ethyl]phosphinic acid; 3-aminopropyl(4,4,4-trifluorobutyl)phosphinic acid; 3-aminopropyl(2-methylthioethyl)phosphinic acid; 3-aminopropyl(methylthiomethyl)phosphinic acid; 3-aminopropyl(2-methylallyl)phosphinic acid; 3-aminopropyl(dodecyl)phosphinic acid; 3-aminopropyl(benzyl)phosphinic acid; 3-aminopropyl(propargyl)-phosphinic acid; 3-aminopropyl(1,3-dithiolan-2-yl)phosphinic acid; 3-aminobutyl(diethoxymethyl)phosphinic acid; 3-amino-1-(p-chlorophenyl)-propyl(diethoxymethyl)phosphinic acid; 3-aminopropyl(di-n-propyloxymethyl)phosphinic acid; 3-aminopropyl(diisopropyloxymethyl)phosphinic acid; 3-aminopropyl(di-n-butyloxymethyl)phosphinic acid; 3-aminopropyl(tetrahydrofuran-2-yl)phosphinic acid; 3-aminopropyl(1-hydroxybutyl)phosphinic acid; 3-aminopropyl(1-hydroxyisobutyl)phosphinic acid; 3-aminopropyl(1-hydroxyethyl)phosphinic acid; 3-aminopropyl(1-hydroxybenzyl)phosphinic acid; 3-aminopropyl(1-hydroxy-4,4,4-trifluorobutyl)phosphinic acid; 3-aminopropyl(1-hydroxy-2-fluoro-(Z)but-2-enyl)phosphinic acid; 3-aminopropyl(1-hydroxy-1-cyclopropylmethyl)phosphinic acid; 3-aminopropyl[1-hydroxy-1-(2-methylthiocyclopropyl)methyl]phosphinic; 3-aminopropyl(1-hydroxy-1-cyclobutylmethyl)phosphinic acid; 3-aminopropyl(−2-hydroxybutyl)phosphinic acid; 3-amiopropyl[2-(R)-hydroxy-3-methylbutyl]phosphinic acid; 3-aminopropyl(1-hydroxycyclobutyl)phosphinic acid; 3-aminopropyl(cyclohexylmethyl)phosphinic acid; 3-aminopropyl(butyl)phosphinic acid; 3-aminopropyl(n-butyl)phosphinic acid; 3-aminopropyl(1-hydroxybutyl)phosphinic acid; 3-aminopropyl(2-hydroxyprop-2-yl)phosphinic acid; 3-aminopropyl-(1,2-dihydroprop-2-yl)phosphinic acid; 3-amino-2-hydroxy-propyl(n-propyl)phosphinic acid; 3-amino-2-(p-chlorophenyl)-propyl(n-propyl)phosphinic acid; 3-amino-1-hydroxy-propyl(n-propyl)phosphinic acid; 3-aminopropyl(4-hydroxybutyl)phosphinic acid; -aminopropyl[2-(S)-methylbutyl]phosphinic acid; 3-aminopropyl(2-hydroxy-3-phthalimido-propyl)phosphinic acid; 3-aminopropyl(3-amino-2-hydroxy-propyl)phosphinic acid; 3-aminopropyl(3-oxobutyl)phosphinic acid; 3-amino-1-hydroxy-propyl(n-butyl)phosphinic acid and its hydrochloride; 3-amino-2-hydroxy-propyl(ethyl)phosphinic acid hydrochloride; 3-Aminopropyl(2-methoxyethyl)phosphinic acid; 3-Aminopropyl(2-ethoxymethyl)phosphinic acid; 3-aminopropyl-(1,1-difluorobutyl)phosphinic acid; 3-amino-2-hydroxy-propyl(n-butyl)phosphinic acid; 3-aminopropyl-(4,4,4-trifluoro-3-methyl-butyl)phosphinic acid; 3-aminopropyl(4,4,4-trifluoro-3-trifluoromethyl-butyl)phosphinic acid; 3-amino-2-(4-chlorophenyl)-propyl(methyl)phosphinic acid; 3-amino-2-(4-fluorophenyl)-propyl(methyl)phosphinic acid; 3-aminopropyl[2-(S)-hydroxy-3-methyl-butyl]phosphinic acid; 3-amino-2-(4-chlorophenyl)-1-hydroxy-propyl(methyl)phosphinic acid; 3-amino-2-(4-chlorophenyl)-1-hydroxy-propyl(n-butyl)phosphinic acid; 3-aminopropyl(2-hydroxy-3-phthalimido-propyl)phosphinic acid; 3-aminopropyl(3-amino-2-hydroxy-propyl)phosphinic acid; or 3-amino-2-hydroxy-propyl(cyclohexylmethyl)phosphinic acid; or a pharmaceutically acceptable salt thereof.

Another specific cognitive enhancing agent useful in the methods of the invention is a compound described in U.S. Pat. Nos. 5,204,342 and 5,132,930. For example, a compound of Formula (II): 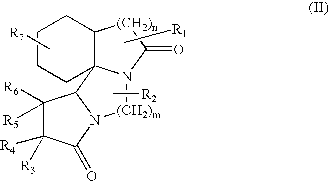
wherein R₁, R₂, R₃, R₅ and R₇ are each, independently of the others, hydrogen or lower alkyl; m is 2 or 3; n is 1 or 2; and either R₄ and R₆ are each hydrogen or R₄ and R₆ together form an additional bond; or a pharmaceutically acceptable salt thereof.
For a Compound of Formula (II) the Following Definitions and Preferred Values Apply:

Since the compounds of Formula (II) contain at least three chiral carbon atoms, they may be, for example, in the form of pure enantiomers, mixtures of enantiomers, such as racemates, pure diastereoisomers, mixtures of diastereoisomers or mixtures of racemates. Preferred compounds of Formula (II) are those having, at the three above-mentioned chiral C-atoms, the stereochemistry of the preferred compounds of Formula (II) described below.

A preferred salt of a compound of Formula (II) is a pharmaceutically acceptable acid addition salt. Such a salt can be formed, for example, with strong inorganic acids, such as mineral acids, for example sulfuric acid, a phosphoric acid or a hydrohalic acid, with strong organic carboxylic acids, such as lower alkanecarboxylic acids, for example acetic acid, saturated or unsaturated dicarboxylic acids, for example malonic, maleic or fumaric acid, or hydroxycarboxylic acids, for example tartaric or citric acid, or with sulfonic acids, such as lower alkanesulfonic acids or unsubstituted or substituted benzenesulfonic acids, for example methane- or p-toluene-sulfonic acid.

Radicals or compounds designated “lower” are to be understood as those having up to and including 7, especially up to and including 4, carbon atoms, unless otherwise specified.

Lower alkyl is C₁-C₄ alkyl, i.e., methyl, ethyl, n-propyl, isopropyl, n-butyl, isobutyl, sec.-butyl or tert.-butyl, and also includes C₅-C₇ alkyl radicals, i.e., corresponding pentyl, hexyl or heptyl radicals.

Halogen is halogen having an atomic number of up to and including 53, i.e., chlorine or bromine, or also fluorine or iodine.

A preferred compound of Formula (II) useful in the methods of the invention is a compound wherein R₁, R₂, R₃, R₅ and R₇ are each, independently of the others, hydrogen or lower alkyl; m is 2 or 3; n is 1 or 2; and R₄ and R₆ are each hydrogen; or a pharmaceutically acceptable salt thereof.

Another preferred compound of Formula (II) useful in the methods of the invention is a compound wherein R₁, R₂, R₃, R₅ and R₇ are each, independently of the others, hydrogen or lower alkyl; m is 2; n is 1; and either R₄ and R₆ are each hydrogen or R₄ and R₆ together form an additional bond; or a pharmaceutically acceptable salt thereof.

Another preferred compound of Formula (II) useful in the methods of the invention is a compound wherein R₁, R₂, R₃, R₅ and R₇ are each, independently of the others, hydrogen or lower alkyl; m is 2; n is 1; and R₄ and R₆ are each hydrogen; or a pharmaceutically acceptable salt thereof.

Another preferred compound of Formula (II) useful in the methods of the invention is a compound wherein R₁, R₂, R₃, R₅ and R₇ are each hydrogen; m is 2; n is 1; and either R₄ and R₆ are each hydrogen or R₄ and R₆ together form an additional bond; or a pharmaceutically acceptable salt thereof.

A more preferred compound of Formula (II) useful in the methods of the invention is a compound wherein R₁, R₂, R₃, R₅ and R₇ are each hydrogen; m is 2; n is 1; and R₄ and R₆ are each hydrogen; or a pharmaceutically acceptable salt thereof.

A most preferred compound of Formula (II) useful in the methods of the invention is the compound, (9aR*,9bR*,13aR*)-2,7-dioxo-1,4,5,7,8,9,9a,10,11,12,13,13a-dodecahydro-2H-pyrrolo[2′,1′:3,4]pyrazino[2,1-i]indole; (9aR*,9bS*,13aS*)-2,7-dioxo-1,4,5,7,8,9,9a,10,11,12,13,13a-dodecahydro-2H-pyrrolo[2′,1′:3,4]pyrazino[2,1-i]indole; (9aR*,9bR*,13aR*)-2,7-dioxo-1,4,5,7,8,9,9a,10,11,12,13,13a-dodecahydro-2H-pyrrolo[2′,1′:3,4]pyrazino[2,1-i]-indole; (9aR*,9bS*,13aS*)-2,7-dioxo-1,4,5,7,8,9,9a,10,11,12,13,13a-dodecahydro-2H-pyrrolo-[2′,1′:3,4]pyrazino[2,1-i]indole; (+)-(9bR*,13aR*)-2,7-dioxo-1,4,5,7,8,10,11,12,13,13a-decahydro-2H-pyrrolo[2′,1′:3,4]pyrazino[2,1-i]indole; (−)-(9bR*,13aR*)-2,7-dioxo-1,4,5,7,8,10,11,12,13,13a-decahydro-2H-pyrrolo-[2′,1′:3,4]pyrazino[2,1-i]indole; (+)-(9aR*,9bR*,13aR*)-2,7-dioxo-1,4,5,7,8,9,9a,10,11,12,13,13a-dodecahydro-2H-pyrrolo[2′,1′:3,4]pyrazino[2,1-i]indole; or (−)-(9aR*,9bR*,13aR*)-2,7-dioxo-1,4,5,7,8,9,9a,10,11,12,13,13a-dodecahydro-2H-pyrrolo[2′,1′:3,4]pyrazino[2,1-i]indole; or a pharmaceutically acceptable salt thereof.

Another specific cognitive enhancing agent useful in the methods of the invention is a compound described in U.S. Pat. No. 5,439,930. For example, a compound of Formula (III): 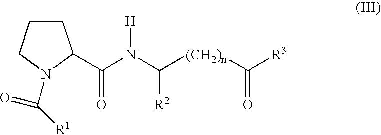
wherein:
R¹=(C₄-C₅)alkyl, cycloalkyl, aralkyl, or aryl;
R²═H₁ (C₁-C₄)alkyl, carbamidoalkyl, or carbalkoxyalkyl;
R³═NH₂, NH(alkyl), N(alkyl)₂, OH, or alkoxy; and
n=0-3, preferably 0-2; or a pharmaceutically acceptable salt thereof.
   For a Compound of Formula (III) the Following Definitions and Preferred Values Apply:

A preferred compound of Formula (III) useful in the methods of the invention is a compound wherein R¹ is selected from the group consisting of isobutyl, pentyl, 1-adamantyl, phenyl, phenylmethyl, and phenylpropyl, and more preferably selected from the group consisting of phenylmethyl and phenyl.

A preferred compound of Formula (III) useful in the methods of the invention is a compound wherein R³ is selected from the group consisting of amino (NH₂), methylamino (NHCH₃), dimethylamino (N(CH₃)), hydroxy (OH), and ethoxy (OC₂H₅), and more preferably selected from the group consisting of amino and ethoxy.

A more preferred compound of Formula (III) useful in the methods of the invention is a compound of Formula (IV):
R¹—C(═O)-L-Pro-Gly-R³ (I)
wherein R¹ is selected from the group consisting of iso-butyl, pentyl, 1-adamantyl, phenyl, phenylmethyl, and phenylpropyl; and R₃ is selected from the group consisting of NH₂, NHCH₃, N(CH₃)₂, OH, and OC₂H₅.

Another more preferred compound of Formula (III) useful in the methods of the invention is the compound: N-phenacetyl-L-prolylglycine ethyl ester; N-phenacetyl-L-prolylglycine amide; N-phenacetyl-L-prolyl-β-alanine ethyl ester; N-phenylacetyl-L-prolyl-β-alanine amide; N-phenylacetyl-L-prolyl-L-aspartic acid diethyl ester; N-phenylacetyl-L-prolyl-L-asparagine amide; N-benzoyl-L-prolylglycine ethyl ester; N-isovaleryl-L-prolylglycine ethyl ester; N-phenylacetyl-L-prolyl-L-valine ethyl ester; N-benzoyl-L-prolyl-L-valine ethyl ester; N-benzoyl-L-prolyl-α-alanine ethyl ester; N-benzoyl-L-prolyl-β-alanine amide; N-benzoyl-L-prolylglycine amide; N-phenylacetyl-L-prolylglycine N-methylamide; N-phenylacetyl-L-prolylglycine dimethylamide; N-phenylacetyl-L-prolyl-L-glutamic acid diethyl ester; N-phenylacetyl-L-prolyl-L-leucine amide; N-phenylacetyl-L-prolylglycine; N-phenylacetyl-L-prolyl-GABA methylester; N-phenylacetyl-L-prolyl-L-alanine ethyl ester; N-caproyl-L-prolylglycine ethyl ester; N-(1-adamantoyl)-L-prolylglycine ethyl ester; or N-phenylbutyl-L-prolyl-glycine ethylester; or a pharmaceutically acceptable salt thereof.

A most preferred compound of Formula (III) useful in the methods of the invention is a compound of Formula (V):
C₆H₅CH₂—C(═O)-L-Pro-Gly-OC₂H₅ (V)
II. Methods of the Invention

Perception of sensory information is encoded in anatomical and temporal patterns of neural activity in the brain. The mental activities associated with thinking, learning, and memory, i.e., cognition, is affected by stress in a number of ways. For example, stress can exacerbate a number of psychiatric disorders which are associated with cognitive deficits (Arnsten and Goldman-Rakic, Arch. Gen. Psychiatry, 55, 362 (1998)). Via the physiological responses that accompany it, stress can alter memory. Brief periods of stress can potentiate or obviate memory formation, i.e., memories when formed may be fragmentary and/or distorted, while severe or prolonged exposure to stressors can have deleterious effects upon broad aspects of cognition including memory.

Physical and psychological stressors provoke the secretion of the catecholamines (epinephrine and norepinephrine) by the sympathetic nervous system, and of the glucocorticoids by the adrenal gland. While catecholamine release can result in enhanced memory for events and increased glucose utilization in the brain, excessive amounts catecholamine are known to disrupt memory. The effects of stress have been attributed to reversible changes in the morphology of neurons within the hippocampus. Truly prolonged exposure to stress can cause irreversible loss of hippocampal neurons, and may be relevant to the cognitive deficits seen in many aged individuals. In addition, stress-induced glucocorticoid levels inhibit both long-term potentiation (LTP) and glucose transport in the hippocampus.

Diseases associated with high glucocorticoid levels have an inhibitory effect on learning and memory. In Aplysia, long-term memory and short-term memory are accompanied by changes in the strength of synaptic connections between the sensory and motor neurons of the gill withdrawal reflex. (Eric R. Kandel et al. eds., Learning and Memory, in Principles of Neural Science, Elsevier Science Publishing Co., Inc., New York, N.Y. (4th ed. 2000). In the long-term as in the short-term process, this increase in synaptic strength again is due to the enhanced transmitter release. There is no change in the sensitivity of the postsynaptic receptor. Serotonin, a modulatory transmitter that produces the short-term facilitation following a single exposure, produces long-term facilitation following four or five repeated exposures. cAMP, the intracellular second messenger involved in the short-term facilitation, also turns on the long-term change. Both cellular studies of Aplysia and genetic studies of Drosophila indicate that the cAMP cascade is important for certain elementary forms of learning and memory storage. There are important differences between the short- and long-term process that emerge on the molecular level. Specifically, whereas short-term facilitation of the synapse between the sensory and motor neurons involves covalent modification of pre-existing proteins and is not affected by inhibitors of protein or RNA synthesis, long-term facilitation requires the synthesis of new protein and mRNA.

Several studies have closely linked declarative knowledge (e.g., that you know something) with the hippocampus and procedural knowledge (e.g., how to do something) with the extrapyramidal systems and the cerebellum. In particular, the hippocampus is associated with storage of declarative memory, and there is evidence that neurons in the hippocampus show plastic capability of the sort that would be required for associative learning. The hippocampus has three major excitatory pathways running from the subiculum to the CA1 region. The perforant pathway runs from the subiculum to the granule cells in the hilus of the dentate gyrus. The axons of the granule cells form a bundle, the mossy fiber pathway, that runs to the pyramidal cells lying in the CA3 region of the hippocampus. Finally, the pyramidal cells in the CA3 region and excitatory collaterals, the Schaeffer collaterals, to the pyramidal cells in the CA1. A brief high-frequency train of stimuli to any one of the three afferent pathways to the hippocampus produces an increase in the excitatory synaptic potential in the postsynaptic hippocampal neurons, which can last for hours, and in the intact animal for days and even weeks. They called this facilitation long-term potentiation (LTP). The axons from the CA3 region of the hippocampus that terminate on the pyramidal cells of the CA1 region use glutamate as their transmitter. Glutamate acts on its target cells in the CA1 region by binding to both N-methyl-D-aspartate (NMDA) and non-NMDA receptors. The non-NMDA receptors dominate in normal synaptic transmission. However, the NMDA receptor-channel, which normally is blocked by Mg²⁺, becomes unblocked and activated when the postsynaptic cell is adequately depolarized by a strong (cooperative) input from many presynaptic neurons. Unblocking the channel allows the influx of Na⁺ and Ca²⁺ into the cell. Research has demonstrated that glucocorticoids increase declarative errors (mistakes on immediate and delayed recall of information) but have no effect on procedural errors (mistakes on serial addition).

The consequences of prolonged exposure to stress of glucocorticoids short-term glucocorticoid exposure leads to increased risk for hippocampal neuron damage. Long-term glucocorticoid exposure leads to hippocampal atrophy. Hippocampal damage is reversible if the exposure to glucocorticoids are limited to certain levels, which implies that there exists a “critical period” of exposure. A brief exposure of glucocorticoids may cause reversible atrophy and loss of dendritic spines in an individual, whereas prolonged glucocorticoid exposure may cause permanent neuronal death.

In addition, nitric oxide (NO) is a well established mediator of memory formation in a variety of species. A number of studies have implicated nitric oxide as a retrograde messenger in hippocampal LTP. Inhibitors of nitric oxide synthase (NOS), or extracellular scavengers of nitric oxide block the induction of LTP. Postsynaptic injections of nitric oxide synthase inhibitors block LTP induction, suggesting nitric oxide is being produced in postsynaptic neurons. Immunochemical studies demonstrated the presence of NOS in the same postsynaptic neurons that undergo LTP. One of the major downstream targets of NO is the activation of soluble guanylyl cyclase. Recent experiments have induced LTP by pairing subthreshold presynaptic stimulation with the application of membrane permeable cGMP analogs, while inhibitors of guanylyl cyclase or cGMP-dependent protein kinases block the induction of LTP.

Prolonged exposure to stress has been linked to cognitive impairments. Long-term glucocorticoid exposure leads to impaired spatial learning. The hippocampal degeneration associated with prolonged glucocorticoid exposure is similar to that seen in aging individuals, i.e., the spatial learning deficits resulting from prolonged glucocorticoid exposure are similar to those seen in aging individuals. Thus, significant amounts of stress can be inhibitory to cognition, while prolonged stress can lead to irreversible hippocampal atrophy.

Thus, a cognitive enhancing agent of the invention can treat cognitive decline by affecting any one of the mechanisms which regulate synaptic plasticity in the brain. The cognitive decline associated with systemic stress is amendable to treatment with the cognitive enhancing agents of the invention.

In one preferred embodiment of the invention, the cognitive enhancing agent is not Ginko, Kava-kava, or Kavain.

The ability of a compound to prevent or treat cognitive change and/or decline associated with systemic stress can be determined using various psychological tests as have been employed in the following studies: New England Journal of Medicine, 344, 395 (2001); Ann. Thorac. Surg., 63, 510 (1997); Crit. Care Med., 28, 1808 (2000); and Lancet, 8, 1601 (1999).

III. Formulations and Routes of Administration of the Cognitive Enhancing Agents of the Invention

The cognitive enhancing agents can be formulated as pharmaceutical compositions and administered to a mammalian host, such as a human patient in a variety of forms adapted to the chosen route of administration, e.g., orally, parenterally, by intravenous, intramuscular, or subcutaneous routes, transdermal (passive or iontophoretic) or via suppository.

Thus, the cognitive enhancing agents may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the active compound may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 60% of the weight of a given unit dosage form. The amount of active compound in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially nontoxic in the amounts employed. In addition, the active compound may be incorporated into sustained-release preparations and devices.

The cognitive enhancing agents may also be administered intravenously or intraperitoneally by infusion or injection. Solutions of the agent or its salt can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the cognitive enhancing agent in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

Useful dosages of the cognitive enhancing agents can be determined by comparing their in vitro activity, and in vivo activity in animal models. Methods for the extrapolation of effective dosages in mice, and other animals, to humans are known to the art; for example, see U.S. Pat. No. 4,938,949. However, the amount of the compound, or an active salt or derivative thereof, required for use in treatment will vary not only with the particular agent selected but also with the route of administration, the nature of the condition being treated and the age and condition of the patient and will be ultimately at the discretion of the attendant physician or clinician.

In general, however, a suitable dose will be in the range of from about 0.5 to about 100 mg/kg, e.g., from about 10 to about 75 mg/kg of body weight per day, such as 3 to about 50 mg per kilogram body weight of the recipient per day, preferably in the range of 6 to 90 mg/kg/day, most preferably in the range of 15 to 60 mg/kg/day.

A suitable dose for a GABA_{β}-receptor antagonist is for example up to about 30 mg/kg/day. Preferably a suitable dose for a GABA_{β}-receptor antagonist will be in the range from about 1 mg/kg/day to about 45 mg/kg/day, more preferably from about 5 mg/kg/day to about 30 mg/kg/day and most preferably from about 10 mg/kg/day to about 20 mg/kg/day.

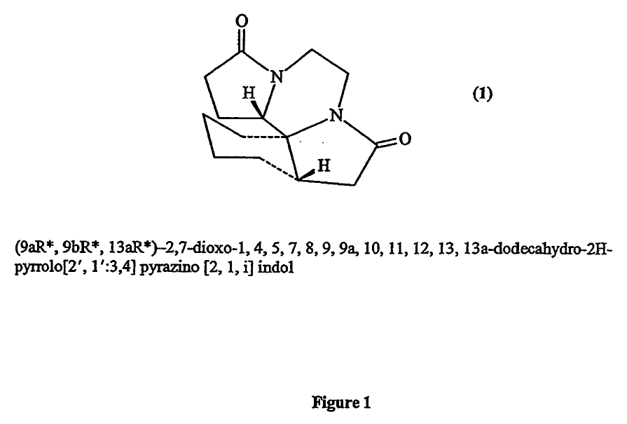

A suitable dose for compound 1 (depicted in FIG. 1) is for example up to about 30 mg/kg/day, preferably about 15 mg/kg/day. Preferably a suitable dose for compound 1 will be in the range from about 1 mg/kg/day to about 45 mg/kg/day, more preferably from about 5 mg/kg/day to about 30 mg/kg/day and most preferably from about 10 mg/kg/day to about 20 mg/kg/day.

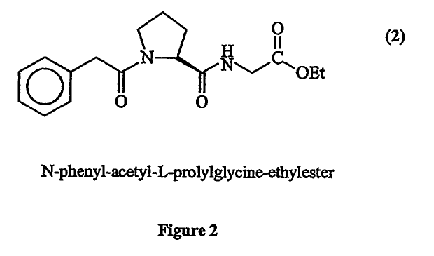

A suitable dose for compound 2 (depicted in FIG. 2) is for example about 0.5 mg/kg/day. Preferably a suitable does for compound 2 will be in the range from about 0.01 mg/kg/day to about 2.0 mg/kg/day, more preferably from about 0.1 mg/kg/day to about 1.0 mg/kg/day and most preferably from about 0.25 mg/kg/day to about 0.75 mg/kg/day.

The compound is conveniently administered in unit dosage form; for example, containing 5 to 1000 mg, conveniently 10 to 750 mg, most conveniently, 50 to 500 mg of active ingredient per unit dosage form.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations.

According to the methods of the invention, one or more cognitive enhancing agents used alone, simultaneously or sequentially can be administered via the preferred route (e.g., orally, parenterally, transcutaneously, rectally, etc.) prior to surgery (e.g. a regimen could begin minutes, hours, or days prior to surgery), during surgery (a single dose or series of doses given over a period of time), or post surgery (e.g. minutes, hours, days, weeks, or months following surgery). In some patients a single course of therapy may produce satisfactory or even optimal results while in other patients a pro-longed regimen or series of episodes of treatment with cognitive enhancing agent(s) may be needed in order that optimal results be realized.

The invention will now be illustrated by the following non-limiting example.

### EXAMPLE 1

Data from a preliminary study in four volunteers suggested that compound 2 is orally bioavailable in humans.

Fourteen healthy young volunteers were orally administered 20 mg/day of compound 2 for 28 days in two Phase I clinical trials. 34 patients with mild to moderate cognitive disturbance were orally administered doses of up to 30 mg/day of compound 2 for 28 days.

After oral administration of the 20 mg/day dose, peak plasma concentrations of 25-38 ng/mL were observed 15-30 minutes after dosing.

### EXAMPLE 2

The efficacy of 10 mg of compound 2 administered orally t.i.d. was investigated in a four-week open-label Phase II clinical trial at two centers. A total of 20 patients with cognitive disturbances secondary to cerebrovascular and traumatic brain injury were orally administered compound 2 during a Phase II clinical trial at a dose of 10 mg t.i.d. for a period of four weeks. At study entry, patients fulfilled specific criteria for mild cognitive disturbance.

Improvement on several tests, including the Mini Mental State Evaluation (MMSE) (Folstein et al., Psychiatric Research, 12, 189-198 (1975)), the Overall Clinical Impression Scale (OCS), which is a system developed by the National Institute of Mental Health: 12-CGI Clinical Global Impression ECDEU Assessment Manual for Psychopharmacology, E. Guyo, ed., Rockville Md., (217-222) (1976), and the Cognitive Capacity Screening Exam (CCSE) (Jacobs et al., Annals of Internal Medicine, 86, 4046 (1977).

Results in the study indicated that over 75% of the treated patients met the definition of efficacy response as determined by a statistically measured improvement in one or more of the tests by the end of the study.

All publications, patents, and patent documents are incorporated by reference herein, as though individually incorporated by reference. The invention has been described with reference to various specific and preferred embodiments and techniques. However, it should be understood that many variations and modifications may be made while remaining within the spirit and scope of the invention.

1. A method of treating cognitive decline associated with systemic stress comprising
(i) identifying a patient as suffering from cognitive decline associated with systemic stress; and,
(ii) administering an effective amount of a cognitive enhancing agent to said patient, wherein said systemic stress is due to surgery, benzodiazapine therapy, or cerebrovascular and traumatic brain injury.

2. The method of claim 1, wherein the cognitive enhancing agent is 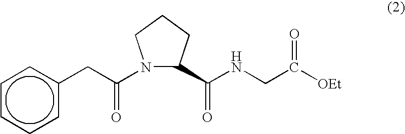
or a pharmaceutically acceptable salt thereof.

3-4. (canceled)

5. The method of claim 2, wherein the systemic stress is due to a medical treatment.

6. The method of claim 5, wherein the medical treatment is surgery.

7. The method of claim 6, wherein the surgery is cardiac surgery.

8. The method of claim 7, wherein the cardiac surgery is CABG.

9. The method of claim 8, wherein the cardiac surgery involves extracorporeal circulation.

10. The method of claim 5, wherein the medical treatment is benzodiazepine therapy.

11. The method of claim 2, wherein the systemic stress is traumatic brain injury.

12. (canceled)

13. The method of claim 1 wherein the cognitive enhancing agent is a compound of Formula I: 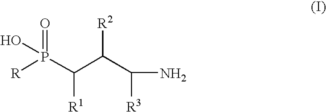
wherein R denotes an aliphatic, cycloaliphatic, cycloaliphatic-aliphatic or araliphatic radical having 2 or more carbon atoms, and wherein one of the groups R¹, R² and R³ represents hydrogen or an aliphatic, cycloaliphatic, araliphatic or aromatic radical, another one of R¹, R² and R³ is hydrogen or, in the case of R¹ and R² is hydroxy, and the remaining one of R¹, R² and R³ is hydrogen, or wherein R denotes methyl, R¹ denotes hydrogen or hydroxy, R² denotes an aromatic radical and R³ represents hydrogen; or a pharmaceutically acceptable salt thereof.

14-33. (canceled)

34. The method of claim 2 wherein the cognitive enhancing agent is, N-phenacetyl-L-prolylglycine ethyl ester; N-phenacetyl-L-prolylglycine amide; N-phenacetyl-L-prolyl-p-alanine ethyl ester; N-phenylacetyl-L-prolyl-p-alanine amide; N-phenylacetyl-L-prolyl-L-aspartic acid diethyl ester; N-phenylacetyl-L-prolyl-L-asparagine amide; N-benzoyl-L-prolylglycine ethyl ester; N-isovaleryl-L-prolylglycine ethyl ester; N-phenylacetyl-L-prolyl-L-valine ethyl ester; N-benzoyl-L-prolyl-L-valine. ethyl ester; N-benzoyl-L-prolyl-p-alanine ethyl ester; N-benzoyl-L-prolyl-p-alanine amide; N-benzoyl-L-prolylglycine amide; N-phenylacetyl-L-prolylglycine N-methylamide; N-phenylacetyl-L-prolylglycine dimethylamide; N-phenylacetyl-L-prolyl-L-glutamic acid diethyl ester; N-phenylacetyl-L-prolyl-L-Ieucine amide; N-phenylacetyl-L-prolylglycine; N-phenylacetyl-L-prolyl-GABA methyl ester; N-phenylacetyl-L-prolyl-L-alanine ethyl ester; N-caproyl-L-prolylglycine ethyl ester; N-(1-adamantoyl)-L-prolylglycine ethyl ester; or N-phenylbutyl-L-prolyl-glycine ethyl ester; or a pharmaceutically acceptable salt thereof.

35-36. (canceled)
